(19) United States
(12) Patent Application Publication
(10) Pub. No.: US 2011/0097780 A1
(43) Pub. Date: Apr. 28, 2011

(54) Method of Xylose Fermentation Having Enhanced Conversion Rate in Fiber Solution

(75) Inventors: Ting-Hsiang Lin, Taoyuan County (TW); Chiung-Fang Huang, Taipei (TW); Gia-Luen Guo, Taipei County (TW); Wen-Song Hwang, Taoyuan County (TW)

(73) Assignee: ATOMIC ENERGY COUNCIL-INSTITUTE OF NUCLEAR ENERGY RESEARCH, Taoyuan County (TW)

(21) Appl. No.: 12/822,274

(22) Filed: Jun. 24, 2010

(30) Foreign Application Priority Data
Oct. 22, 2009 (TW) 098135724

(51) Int. Cl. C12P 7/10

(52) U.S. Cl. 435/165

(57) ABSTRACT

A method is provided for improving the efficiency of xylose fermentation in lignocellulosic hydrolysate. The disclosed embodiment raises the efficiency of xylose conversion by adding a specific lignocellulosic material during fermentation. In particular, a 10% enhancement in the efficiency of xylose conversion for ethanol production was given, and the ethanol yield is achieved 90% after adding the specific lignocellulosic material.

### CROSS REFERENCE TO RELATED PATENT APPLICATIONS

This application claims priority from Taiwan Patent Application No. 098135724, filed in the Taiwan Patent Office on Oct. 22, 2009, and incorporates the Taiwan patent application in its entirety by reference.

### TECHNICAL FIELD OF THE DISCLOSURE

The present disclosure relates to xylose fermentation; more particularly, relates to adding a lignocellulosic material in a xylose fermentation process for improving a xylose conversion rate of 10% and thus achieving an ethanol yield above 90%.

### DESCRIPTION OF THE RELATED ARTS

Bio-ethanol, a potential fuel for replacing gasoline, is mainly obtained from starch and sucrose in cereal and cane respectively by fermentation processes using yeast after some simple preparation.

Yet, ethanol production from grain may compete for the staple food supply. A true story is that the prices of grains in year 2008 are raised rapidly due to the increase of amounts in bioethanol production with the grain as the raw materials. Hence, non-grain biomaterials like wood, bagasse, rice straw, corn stover, wheat straw, silvergrass, corn cob, paper waste etc. are good alternatives as the feedstock for ethanol production because the lignocellulosic biomass have the advantages of great amount, wide varieties and non-conflict with grain storage.

In general, lignocellulosic materials contains 60˜80% of cellulose and hemicellulose and 15˜25% of lignin. Cellulose and hemicellulose is mainly formed through the polymerization of glucose and C5 sugars including xylose and arabrose. For obtaining ethanol, hemicellulose is usually decomposed into xylose through a thermal chemical pretreatment like diluted-acid hydrolysis or acid-catalyzed steam explosion.

Basically, a certain ratio of biomass is mixed into a solution to be put into a reactor. Then 1% to 2% (w/w) of diluted-acid is added for reaction with a few minutes. The xylose released into solution and thus obtained a so-called hydrolysates, However, the xylose-rich hydrolysates for fermentation has the problem with high concentration of sulfate ion, which may affects the ability of yeast on converting xylose to ethanol. Moreover, the diluted-acid hydrolysis pretreatment may simultaneously generates fermentation inhibitors like acetic acid, furfural, hydroxymethyl furfural, etc.

Since general the wine yeasts do notare unable to convert xylose into ethanol efficiently, and only a few species of yeasts having the capacity for converting xylose into ethanol are lacked. Native organisms such as Pichia Stipitis and Candida shehatae canhas been reported to show the capacity for convert xylose into ethanol withby lignocellulosic solutions hydrolysates, which are made from corn stover, rice straw, hard wood, soft wood, corn cob, water hyacinth, wheat straw, sunflower seed hull, etc. However, as reported in “Production of ethanol from corn stover hemicelluloses hydrolyzate using Pichia stipitis. Journal of Industrial Microbiology and Biotechnology 34, 723-727, by Agbogbo, F. K. and K. S. Wenger, 2007, a xylose fermentation of corn stover hydrolysates may give an ethanol yield of 85% by Pichia Stipitis, while the other reports only obtains ethanol yields below 75%. Yet, these ethanol yields are not high enough for massive production of ethanol. Hence, the prior arts do not fulfill all users' requests on actual use.

### SUMMARY OF THE DISCLOSURE

The main purpose of the present disclosure is to add a lignocellulosic material in a xylose fermentation process for improving a xylose conversion efficiency of 10% and thus achieving an ethanol yield above 90%.

The second purpose of the present disclosure is to improve a conversion of a non-detoxified xylose-rich hydrolysates for giving 1.1 to 1.6 times of ethanol productivity.

The third purpose of the present disclosure is to add a lignocellulosic material for a fast co-precipitating with yeast and make fermentation available in a repeated batch while the yeast is recyclable.

To achieve the above purposes, the present disclosure is a method of enhanced ethanol production in xylose fermentation using lignocellulosic hydrolysates., comprising steps of: (a) obtaining a raw material; (b) mixing the raw material with a solution of diluted sulfuric acid to obtain a mixed solution; (c) boiling the mixed solution and then removing liquid portion from the mixed solution to obtain solid portion of the mixed solution through a process of solid-liquid separation; (d) adding a solution having cellulase with into the solid portion for processing a reaction to removing cellulose in the solid portion; (e) after removing the cellulose in the solid portion, separating the solid portion from the solution to obtain a lignocellulosic material; and (f) adding the lignocellulosic material into a solution having xylose to process a fermentation with a yeast to obtain ethanol. Accordingly, a novel method of xylose fermentation having enhanced conversion efficiency in lignocellulosic hydrolysates is achieved.

### BRIEF DESCRIPTIONS OF THE DRAWINGS

The present disclosure will be better understood from the following detailed description of the preferred embodiment according to the present disclosure, taken in conjunction with the accompanying drawings, in which

FIG. 1 is the flow chart showing the preferred embodiment according to the present disclosure;

FIG. 2 is the illustration showing the xylose conversion efficiency in the detoxified hydrolysates;

FIG. 3 and FIG. 4 are the illustrations showing the xylose conversion efficiency in the non-detoxified hydrolysates at pH5.0 and pH6.0;

FIG. 5 is the illustration showing the xylose conversion efficiency in the non-detoxified hydrolysates obtained from acid-catalyzed reaction at 160° C.

FIG. 6 is the illustration showing the xylose conversion efficiency in the detoxified lignocellulosic hydrolysates obtained from acid-catalyzed reaction at 200° C.

### DESCRIPTION OF THE PREFERRED EMBODIMENT

The following description of the preferred embodiment is provided to understand the features and the structures of the present disclosure.

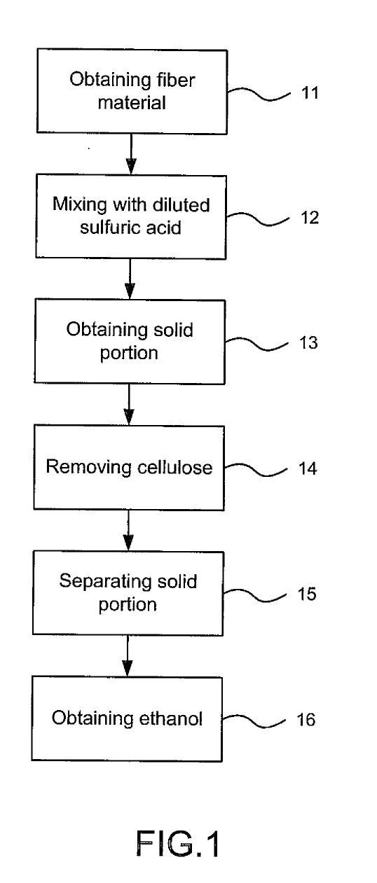

Please refer to FIG. 1, which is a flow chart showing a preferred embodiment according to the present disclosure. As shown in the figure, the present disclosure is a method of xylose fermentation having enhanced conversion efficiency in lignocellulosic hydrolysates, where a lignocellulosic material is added during xylose fermentation to increase a xylose conversion rate for 10% and to obtain a final ethanol conversion rate above 90%. The present disclosure comprises the following steps:

(a) Obtaining raw material 11: A lignocellulosic material is prepared at first for xylose fermentation. The lignocellulosic material is made from a raw material, like corn stover, rice straw, hard wood, corn cob, water hyacinth, wheat straw or sunflower seed hull.

(b) Mixing with diluted sulfuric acid 12: The raw material is mixed with a solution of diluted sulfuric acid, where the solution has a concentration of diluted sulfuric acid between 1% and 3%; a ratio of the raw material to the solution at weight basis is between 1:5 and 1:10;

(c) Obtaining solid portion 13: The mixed solution is boiled at a temperature between 130° C. and 200° C. for 1-15 minutes. Then solution portion of the mixed solution is removed to obtain solid portion of the mixed solution through a process of solid-liquid separation.

(d) Removing cellulose 14: A solution having cellulase is added into the solid portion to process for 72 hours and then remove cellulose in the solid portion.

(e) Separating solid portion 15: After removing the cellulose in the solid portion, the solid portion is separated from the solution to obtain a lignocellulosic material.

(f) Obtaining ethanol 16: The lignocellulosic material is then added into a lignocellulosic hydrolysates having xylose to process fermentation with a yeast to obtain ethanol, where the yeast is capable of decomposing xylose, like Pichia Stipitis; a ratio of the lignocellulosic material to the hydrolysates having xylose at weight basis is between 1% and 5%; the hydrolysates having xylose has a pH value between 5.0 and 7.0; and the fermentation is processed with an amount of air injection between 0.01 vvm and 0.05 vvm.

Thus, a novel method of xylose fermentation having enhanced conversion efficiency in lignocellulosic hydrolysates is obtained.

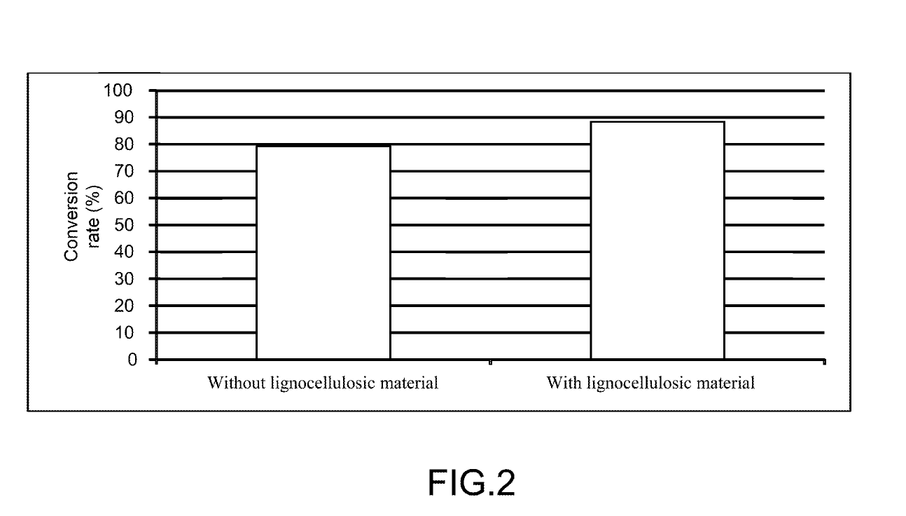

Please refer to FIG. 2, which is a illustration showing a xylose conversion efficiency of a detoxified hydrolysates. As shown in the figure, a hydrolysate made form lignocelluloses processed through acid hydrolysis is obtained. The hydrolysates are detoxified through overliming and then to be fermented with a lignocellulosic material added;, the final solution thus obtained containing 1.9 g/L of acetic acid, 20 g/L of xylose and 2.6 g/L of glucose. Therein, the hydrolysate is fermented at 30° C. with an agitation rate at 100 rpm and yeast added for fermentation has a volumetric ratio to the solution as 1:6 (v/v). As a result shows, when a ratio of the lignocellulosic material to the hydrolysates at weight basis is 1:20, the ethanol yield of the hydrolysates detoxified through overliming is increased for 10%.

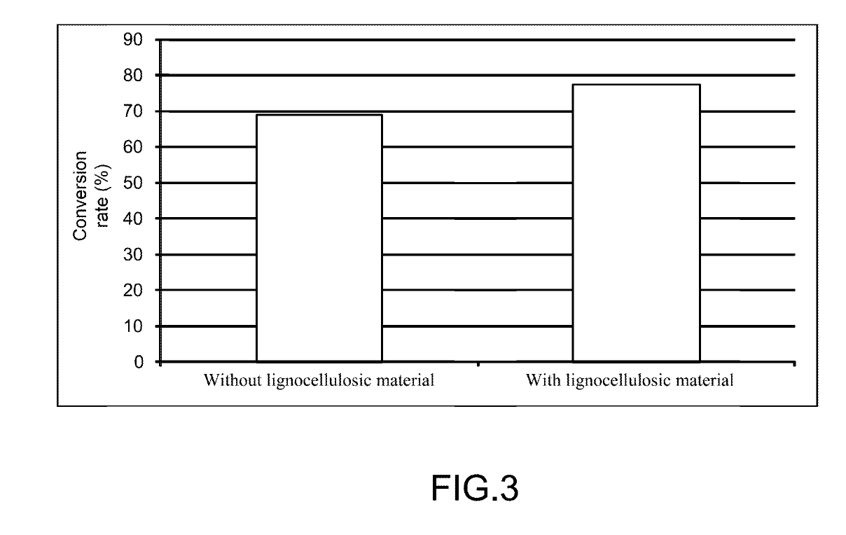

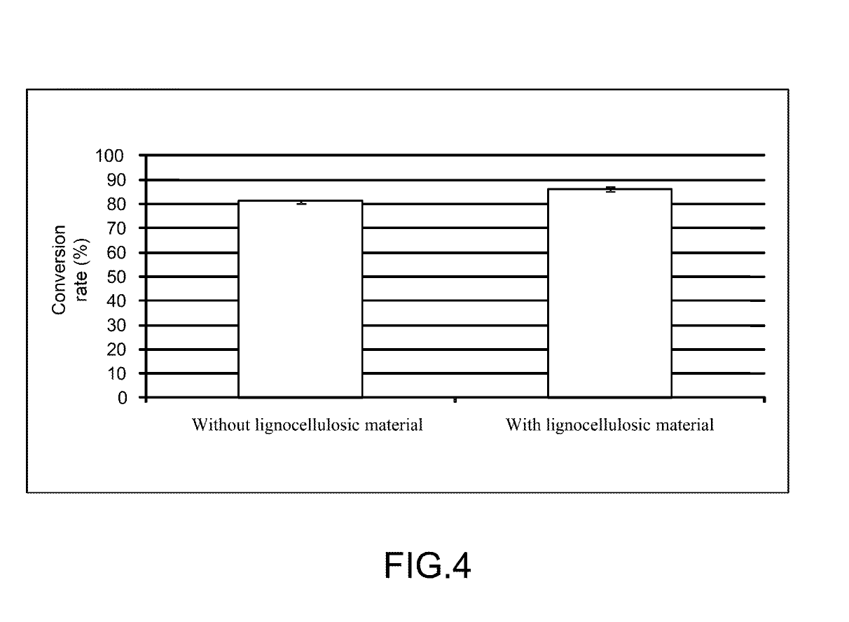

Please refer to FIG. 3 and FIG. 4, which are illustrations showing xylose conversion efficiency of a non-detoxified hydrolysates at pH5.0 and pH6.0 respectively. As shown in the figures, a non-detoxified solution obtained though acid-catalyzed reaction at 130° C. was fermented at various pH conditions. Therein, the volume of the solution is 50 mL; the solution contains 1.9 g/L of acetic acid, 20 g/L of xylose and 2.6 g/L of glucose; the solution is fermented at 30° C. with an agitation rate at 100 rpm; and, yeast added for fermentation has a volumetric ratio to the solution as 1:6 (v/v). As results shows, both xylose conversion efficiency are increased when the pH values for the fermentation are controlled at pH5.0 and pH6.0 with 2.5 grams of lignocellulosic material added. In general, when the pH value is 5.0, the concentration of acetic acid is 1.9 g/L. The growth of the yeast and yield of ethanol are thus inhibited by acetic acid at this concentration and then the ethanol yield is only 68%. Yet, after the lignocellulosic material was added, the ethanol yield is increased for 10% and reaches 77%. When the pH value is raised to 6.0, because acetic acid is dissociated, the circumstance for fermentation becomes better. Yet, after the lignocellulosic material is added, the ethanol yield is still increased for 5%. Thus is proved that the lignocellulosic material added can improve the ethanol yield at the even worse circumstance for fermentation.

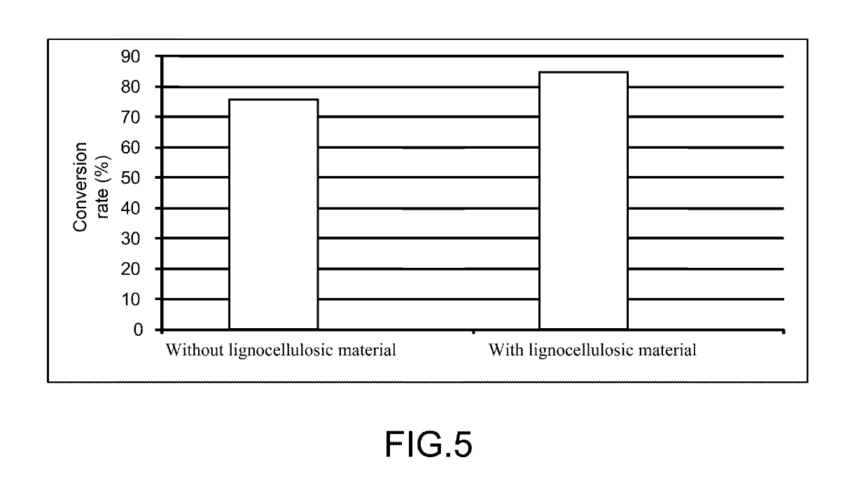

Please refer to FIG. 5, which is a illustration showing a xylose conversion efficiency of a non-detoxified hydrolysates obtained form acid-catalyzed reaction at 160° C. As shown in the figure, a non-detoxified hydrolysates is obtained to be fermented from acid-catalyzed reaction at 160° C. for 30 minutes. Therein, the hydrolysates contains 1.7 g/L of acetic acid, 25 g/L of xylose, 8 g/L of glucose, 0.2 g/L of furfural and 0.4 g/L of formic acid; the hydrolysates was fermented at 30° C. with an agitation rate at 100 rpm; and, Pichia Stipitis was added for fermentation with a volumetric ratio to the solution as 1:6 (v/v). When the lignocellulosic material was added into the solution with a ratio of 1:20 at weight basis, the ethanol yield then increased for 10%.

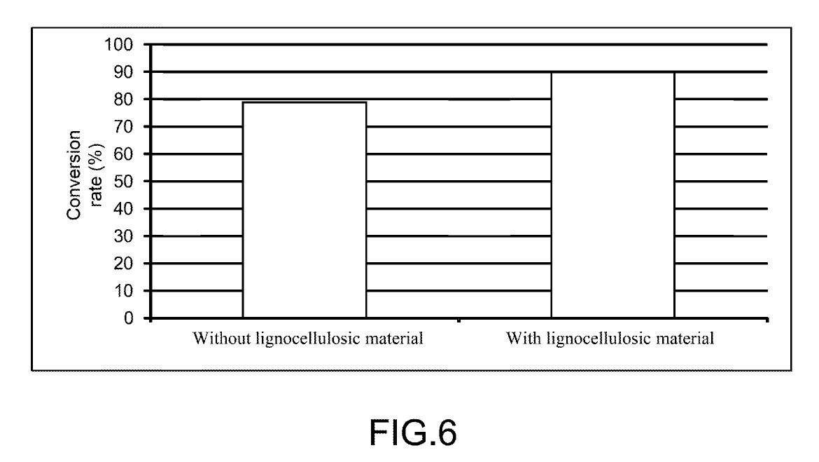

Please refer to FIG. 6, which is a illustration showing a xylose conversion efficiency of a detoxified hydrolysates. As shown in the figure, a detoxified solution is obtained to be fermented in a 5-liter fermentation tank from at 200° C. thermal chemical reaction for 1 minute. Then, using overliming for detoxicification; the detoxified solution contains 1.7 g/L of acetic acid, 20 g/L of xylose, 7 g/L of glucose, 0.2 g/L of furfural, 0.1 g/L of hydroxymethylfurfural (HMF) and 0.4 g/L of formic acid; the solution is fermented at 30° C. with agitation rate at 100 rpm; and Pichia stipitis was added for fermentation with a volumetric ratio to the solution as 1:6 (v/v). When the lignocellulosic material added into the solution with ratio of 1:20 at weight basis, the ethanol yield is increased to 93%.

Thus, the present disclosure is used to ferment a non-detoxified xylose-rich hydrolysates with an improved ethanol productivity for 1.1 to 1.6 times, when the hydrolysates was made by a dilute acid process at 130° C. to 200° C. Besides, the present disclosure uses xylose-fermented yeast for a fast co-precipitation with the lignocellulosic materials added also causes recycling the yeast in a repeated batch to be available.

To sum up, the present disclosure is a method of xylose fermentation having enhanced conversion efficiency in lignocellulosic hydrolysates, where a lignocellulosic material is added in a process of xylose fermentation for improving an ethanol production for 10% and thus achieving an ethanol yield above 90%.

The preferred embodiment herein disclosed is not intended to unnecessarily limit the scope of the disclosure. Therefore, simple modifications or variations belonging to the equivalent of the scope of the claims and the instructions disclosed herein for a patent are all within the scope of the present disclosure.

What is claimed is:

1. A method of xylose fermentation having enhanced conversion efficiency in lignocellulosic hydrolysates, the method comprising:
obtaining a raw material;
mixing raw material with a solution of diluted sulfuric acid to obtain a mixed solution,
wherein said solution has a concentration of diluted sulfuric acid between 1% and 3%; and
wherein a ratio of said raw material to said solution at weight basis is between 1:5 and 1:10;
boiling said mixed solution and then removing solution portion of said mixed solution to obtain solid portion of said mixed solution through a process of solid-liquid separation, wherein said mixed solution is boiled at a temperature between 130° C. and 200° C. for a period of time between 1 and 15 minutes;
adding a solution having cellulase into said solid portion to process for removing cellulose in said solid portion, wherein said reaction is processed for 72 hours;
after removing said cellulose in said solid portion, separating said solid portion from said solution to obtain a lignocellulosic material; and
adding said lignocellulosic material into a solution having xylose to process fermentation with a yeast to obtain ethanol.

2. The method according to claim 1, wherein said raw material is selected from a group consisting of corn stover, rice straw, hard wood, corncob, water hyacinth, wheat straw and sunflower seed hull.

3. The method according to claim 1, wherein said yeast is a yeast capable of decomposing xylose.

4. The method according to claim 3, wherein said yeast is Pichia Stipitis.

5. The method according to claim 1, wherein said solution having xylose has a pH value between 5.0 and 7.0.

6. The method according to claim 1, wherein said fermentation is processed with an amount of air injection between 0.01 vvm and 0.05 vvm.

7. The method according to claim 1, wherein a weight ratio of said lignocellulosic material to that of said solution having xylose is between 1% and 5%.
